(19) United States
(12) Patent Application Publication
(10) Pub. No.: US 2019/0040066 A1
(43) Pub. Date: Feb. 7, 2019

(54) PROCESS FOR THE PREPARATION OF PHOSPHATIDYLINOSITOL 3-KINASE INHIBITOR

(71) Applicant: Lupin Limited, Mumbai (IN)

(72) Inventors: Sanket Pandurang Jadhav, Pune (IN); Dipak Vasant Patil, Pune (IN); Deepak Puna Mahajan, Pune (IN); Sagar Purushottam Nehate, Pune (IN); Himanshu Madhav Godbole, Pune (IN); Girij Pal Singh, Pune (IN)

(21) Appl. No.: 16/075,003

(22) PCT Filed: Feb. 3, 2017
(86) PCT No.: PCT/IB2017/050582

§ 371 Date: Aug. 2, 2018

(30) Foreign Application Priority Data
Feb. 3, 2016 (IN) 201621003903

(51) Int. Cl. C07D 473/34

(52) U.S. Cl. CPC C07D 473/34

(57) ABSTRACT

The present invention relates to processes for the preparation of Phosphatidylinositol 3-Kinase Inhibitor (PI3K) compound of formula-1 via novel intermediates (I).

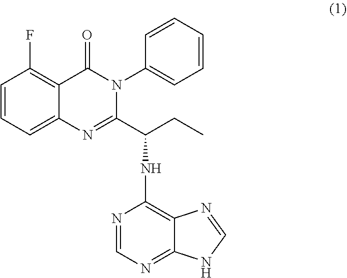 [SMILES: CC[C@H](Nc1ncnc2[nH]cnc12)c1nc2cccc(F)c2c(=O)n1-c1ccccc1]

### FIELD OF THE INVENTION

The present invention relates to processes for the preparation of Phosphatidylinositol 3-Kinase Inhibitor (PI3K) such as Idelalisib, the compound of formula-1 via novel intermediates.

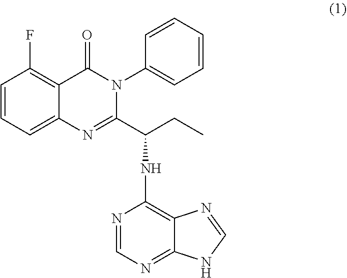 [SMILES: CC[C@H](Nc1ncnc2[nH]cnc12)c1nc2cccc(F)c2c(=O)n1-c1ccccc1]

The chemical name for Idelalisib is 5-fluoro-3-phenyl-2-[(1S)-1-(9H-purin-6-ylamino)propyl]quinazolin-4(3H)-one. Idelalisib has molecular formula of C₂₂H₁₈FN₇O and molecular weight of 415.42 gm/mol.

### BACKGROUND OF THE INVENTION

Idelalisib is used for the treatment of chronic lymphocytic leukemia (CLL), follicular B-cell non-Hodgkin lymphoma (FL) and small lymphocytic lymphoma (SLL). The substance acts as a phosphoinositide 3-kinase inhibitor; more specifically, it blocks P110δ, the delta isoform of the enzyme phosphoinositide 3-kinase.

The processes for the preparation of Idelalisib have been disclosed in applications, WO2005113554, CN104130261, CN104262344 and WO2015095601. Each of these references is hereby incorporated herein by way of reference in its entirety.

There is a need to develop inexpensive and alternative process in making such PI3K inhibitors such as Idelalisib of formula-1. Hence, it is found that the present invention meets this objective and thus provides a process that is industrially advantageous.

### OBJECT OF THE INVENTION

The present invention relates to the synthesis or preparation of Phosphatidylinositol 3-Kinase Inhibitor (PI3K).

In another aspect, the present invention provides new process for the synthesis or preparation of Idelalisib, the compound of formula-1.

In another aspect, the present invention provides novel synthetic intermediates of the above-mentioned process.

The present invention further relates to the use of Idelalisib prepared by the process of present invention in the treatment for PI3K-mediated disorders such as cancer.

### DETAILED DESCRIPTION OF THE INVENTION

The present invention relates to a process for the preparation of Phosphatidylinositol 3-Kinase Inhibitor.

In one embodiment, the present invention relates to a process for the preparation of Idelalisib, the compound of formula-1 and its synthetic intermediates.

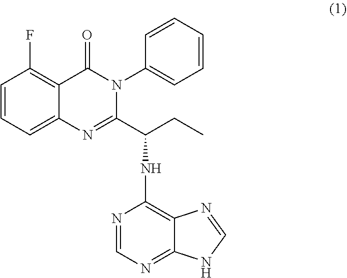 [SMILES: CC[C@H](Nc1ncnc2[nH]cnc12)c1nc2cccc(F)c2c(=O)n1-c1ccccc1]

In one embodiment, the present invention provides a process for the preparation of formula-5

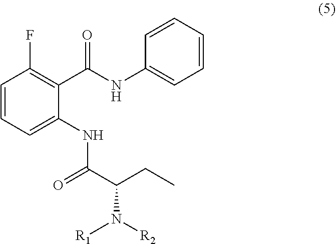

or a salt thereof, comprising reacting a compound of formula-3

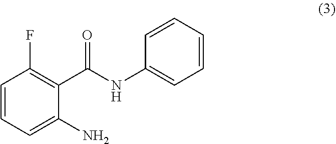 [SMILES: Nc1cccc(F)c1C(=O)Nc1ccccc1]

or a salt thereof, and a compound of formula-4

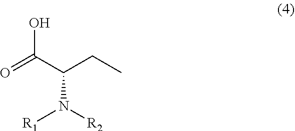

wherein, the compound of formula-5 or a salt thereof is obtained and wherein R₁ and R₂ comprise independently hydrogen and an amino protective group.

In another embodiment, the process further comprises reacting the compound of formula-5 or a salt thereof, wherein at least one of R₁ and R₂ of the compound of formula-5 comprises an amino protective group with deprotection reagent to remove the amino protective group; wherein a compound of formula-6

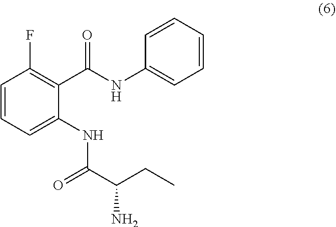 [SMILES: CC[C@H](N)C(=O)Nc1cccc(F)c1C(=O)Nc1ccccc1]

or a salt thereof is obtained.

In some embodiments, the process further comprises reacting the compound of formula-6 or a salt thereof with a compound of formula-7 or formula-7′

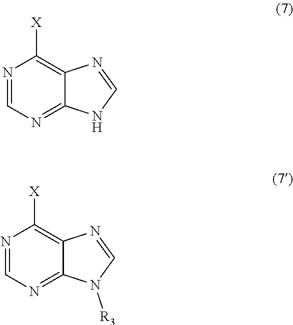

wherein, X comprises halogen, mesylate (methanesulfonate) and tosylate (p-toluene sulfonate) and R₃ comprises an amino protective group and wherein a compound of formula-8 or a compound of formula-8′

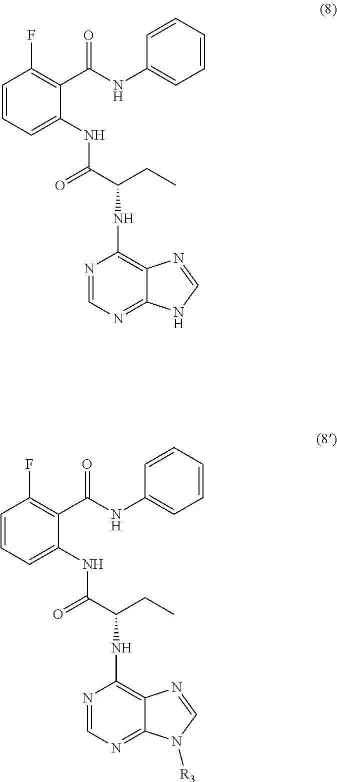

or a salt thereof is obtained.

In some embodiments, when the compound of formula-8′ is formed, the process further comprises reacting the compound of formula-8′ or a salt thereof with deprotection reagent to remove the amino protective group, wherein the compound of formula-8

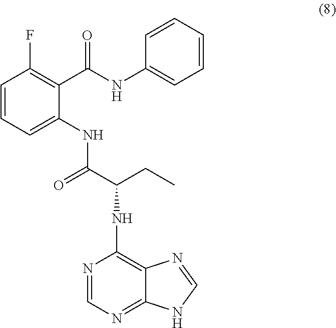 [SMILES: CC[C@H](Nc1ncnc2[nH]cnc12)C(=O)Nc1cccc(F)c1C(=O)Nc1ccccc1]

or a salt thereof is obtained.

In some embodiments, the process further comprises the compound of formula-8 or a salt thereof with cyclization reagent to cyclize the compound of formula-8, wherein the compound of formula-1

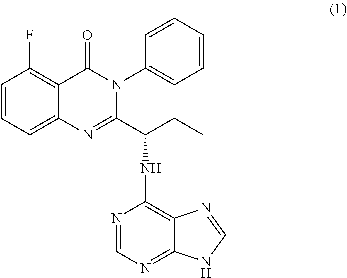 [SMILES: CC[C@H](Nc1ncnc2[nH]cnc12)c1nc2cccc(F)c2c(=O)n1-c1ccccc1]

or a salt thereof is obtained.

In some embodiments, the present invention relates to novel intermediate compounds formed from the processes disclosed herein. In some embodiments, the invention of the application relates to the novel compounds, viz. the compound formula-6, the compound formula-8 and the compound formula-8′ or salts thereof.

In one more embodiment, alternatively the present invention relates to a process for the preparation of a compound of formula-1

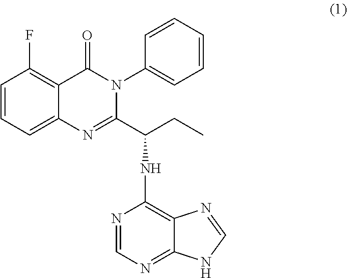 [SMILES: CC[C@H](Nc1ncnc2[nH]cnc12)c1nc2cccc(F)c2c(=O)n1-c1ccccc1]

or a salt thereof, which comprises
step (i) reacting the compound of compound of formula-8′

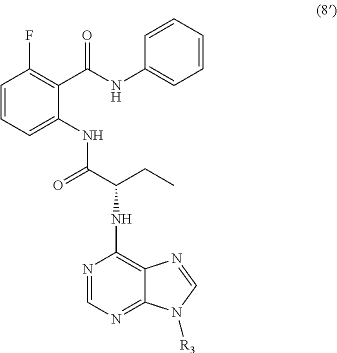

or a salt thereof with cyclization reagent to cyclize the compound of formula-8′, wherein the compound of formula-14

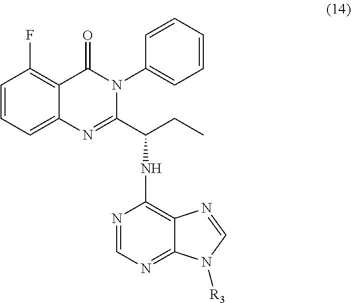

or a salt thereof is obtained; and
step (ii) reacting the compound of formula-14 or a salt thereof with deprotection reagent to remove the amino protective group, wherein the compound of formula-1 is obtained.

In one more embodiment, the present invention relates to a process for the preparation of a compound of formula-10

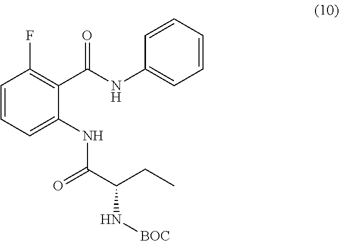 [SMILES: CC[C@H](NC(=O)OC(C)(C)C)C(=O)Nc1cccc(F)c1C(=O)Nc1ccccc1]

or a salt thereof, comprising reacting a compound of formula-3

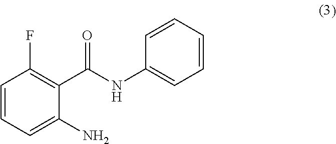 [SMILES: Nc1cccc(F)c1C(=O)Nc1ccccc1]

or a salt thereof, with a compound of formula-9

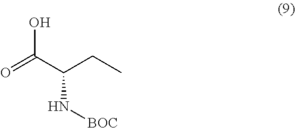

wherein, the compound formula-10 or a salt thereof is obtained.

In some embodiments, the process further comprises reacting the compound of formula-10 or a salt thereof with an acid; wherein a compound of formula-11

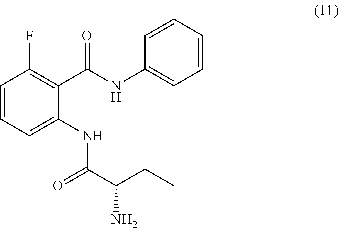 [SMILES: CC[C@H](N)C(=O)Nc1cccc(F)c1C(=O)Nc1ccccc1]

or a salt thereof is obtained.

In some embodiments, the process further comprises reacting the compound of formula-11 or a salt thereof with a compound of formula-12 or a compound of formula-12′

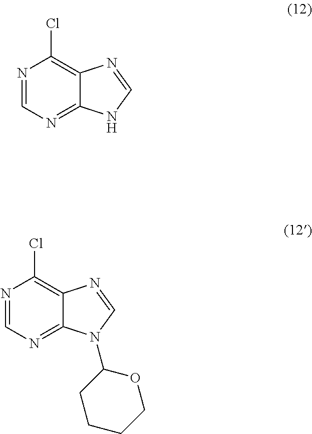

wherein, a compound of formula-13 or a compound of formula-13′

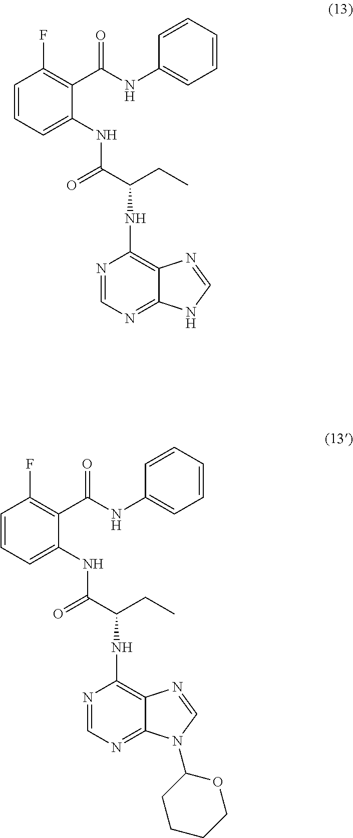 [SMILES: CC[C@H](Nc1ncnc2[nH]cnc12)C(=O)Nc1cccc(F)c1C(=O)Nc1ccccc1 ; CC[C@H](Nc1ncnc2c1ncn2C1CCCCO1)C(=O)Nc1cccc(F)c1C(=O)Nc1ccccc1]

or a salt thereof is obtained.

In some embodiments, when the compound of formula-13′ is obtained, the process further comprises reacting the compound of formula-13′ or a salt thereof with an acid to remove amino protective group of formula-13′, wherein the compound of formula-13

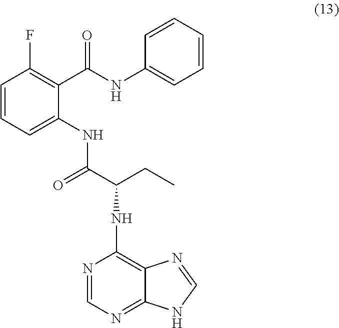 [SMILES: CC[C@H](Nc1ncnc2[nH]cnc12)C(=O)Nc1cccc(F)c1C(=O)Nc1ccccc1]

or a salt thereof is obtained.

In some embodiments, the process further comprises reacting the compound of formula-13 or a salt thereof with Bis(trimethylsilyl)acetamide, wherein compound of formula-1

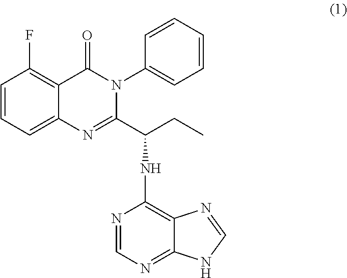 [SMILES: CC[C@H](Nc1ncnc2[nH]cnc12)c1nc2cccc(F)c2c(=O)n1-c1ccccc1]

or a salt thereof is obtained.

In one more embodiment, alternatively the present invention relates to a process for the preparation of a compound of formula-1

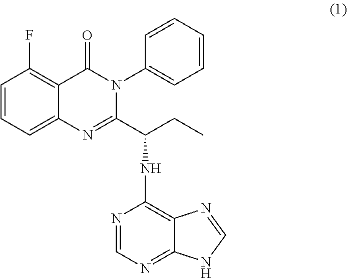 [SMILES: CC[C@H](Nc1ncnc2[nH]cnc12)c1nc2cccc(F)c2c(=O)n1-c1ccccc1]

or a salt thereof comprising
step (i) reacting the compound of formula-13′

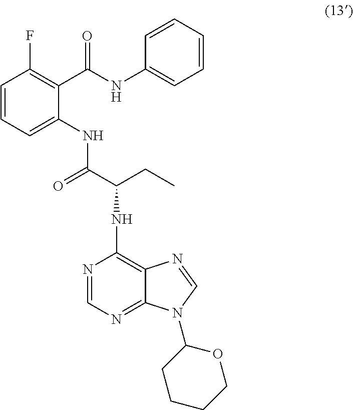 [SMILES: CC[C@H](Nc1ncnc2c1ncn2C1CCCCO1)C(=O)Nc1cccc(F)c1C(=O)Nc1ccccc1]

or a salt thereof with cyclization reagent to cyclize the compound of formula-13′ to obtain a compound of formula-14′,

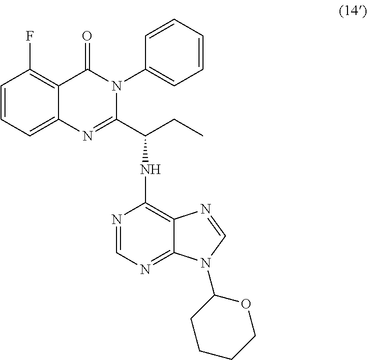 [SMILES: CC[C@H](Nc1ncnc2c1ncn2C1CCCCO1)c1nc2cccc(F)c2c(=O)n1-c1ccccc1]

or a salt thereof; and
step (ii) reacting the compound of formula-14′ or a salt thereof with deprotection reagent to remove the amino protective group, wherein the compound of formula-1 or a salt thereof is obtained.

The present invention is schematically represented by the following scheme-1:

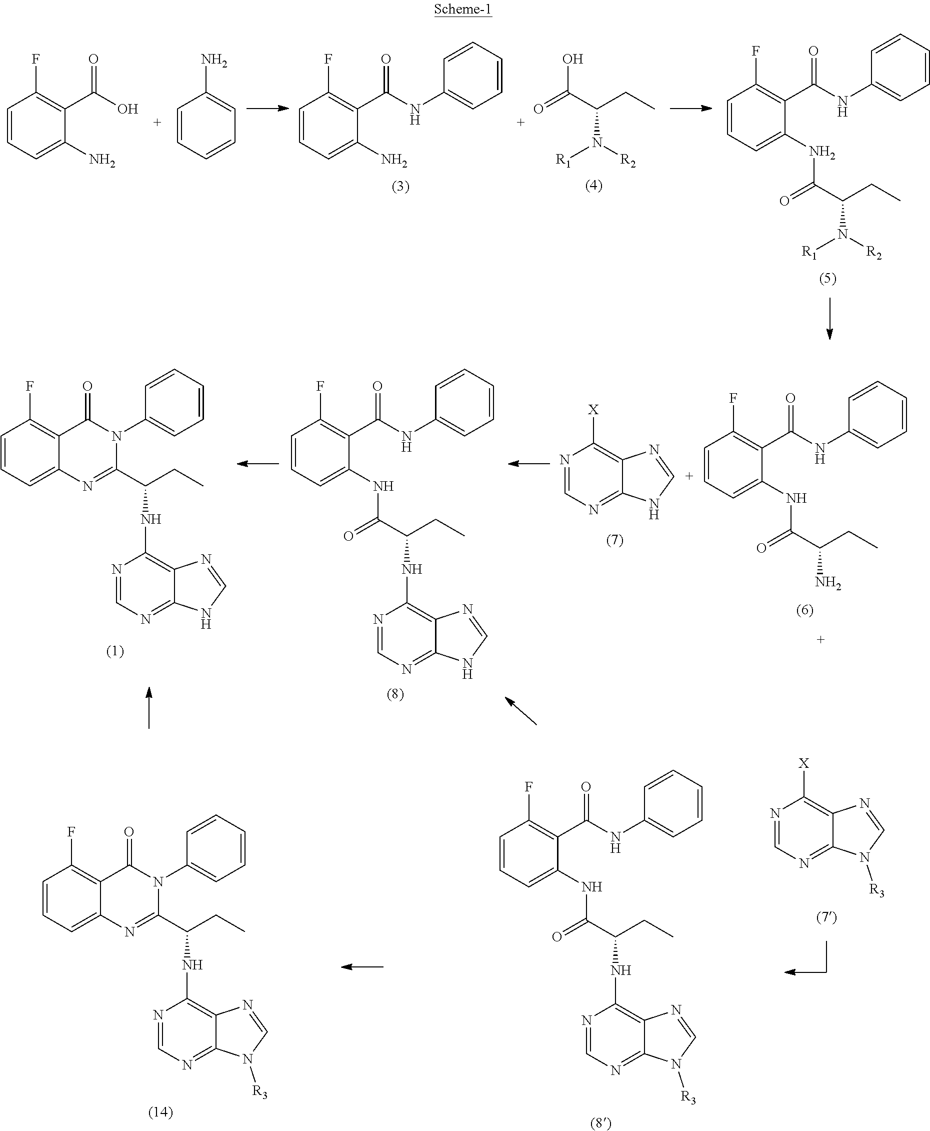

### Abbreviations

BOC: tert-Butyl carbamate,

### CDI: 1,1′-Carbonyldiimidazole,

DABCO: 1,4-Diazabicyclo[2.2.2]octane,
DBU: 1,8-Diazabicyclo[5.4.0]undec-7-ene,

### DCC: N,N′-Dicyclohexylcarbodiimide,

### DIC: N,N′-Diisopropylcarbodiimide,

### DPP: Diphenylphosphite,

EDC: N-(3-Dimethylaminopropyl)-N′-ethylcarbodiimidehydrochloride,
FMOC: 9-Fluoroenylmethyl carbamate,
H₂SO₄: Sulfuric acid,
H₃BO₃: Boric acid,
H₃PO₄: Phosphoric acid,
HBr: Hydrobromic acid,
HCl: Hydrochloric acid,
HClO₄: Perchloric acid,
HF: Hydrofluoric acid,
HNO₃: Nitric acid,

### NMI: N-methylimidazole

TBAF: Tetra-n-butylammonium fluoride,
TFA: Trifluoroacetic acid

### THP: Tetrahydropyran,

### TPP: Triphenylphosphite.

### Definitions

The “amino protective group” comprises suitable nitrogen protecting groups including amino, amido or imino protecting groups which are conventionally used in organic chemistry and/or peptide synthesis or the groups described in the relevant chapters of standard reference works such as J. F. W. McOmie, “Protective Groups in Organic Chemistry”, Plenum Press, London and New York 1973 or T. W. Greene and P. G. M. Wuts, “Protective Groups in Organic Synthesis”. The preferred “amino protective group” generally comprise carbamate based amino protective groups, tetrahydropyranyl group or alkylsilyl groups.

Non-limiting examples of carbamate based amino protective groups include protective groups based on methyl carbamate, 9-fluoroenylmethyl carbamate, 2,2,2-trichloroethyl carbamate, 2-trimethylsilylethyl carbamate, 1,1-dimethylpropynyl carbamate, t-butyl carbamate, vinyl carbamate and allyl carbamate. In another embodiment, the amino protective group comprises alkylsilyl groups selected from trialkyl silyl groups such as trimethyl silyl, tert-butyldimetylsilyl and triethyl silyl group.

The term “deprotection reagent” comprises the conventionally used in organic chemistry and/or peptide synthesis for the deprotection of amino protective group from the amino group. The deprotection reagent comprises acid, wherein the acid comprises organic acid or inorganic acid. The term “acid” comprises hydrochloric acid, nitric acid, phosphoric acid, sulfuric acid, boric acid, hydrofluoric acid, hydrobromic acid, and perchloric acid.

The term “dehydrating reagent” comprises suitable dehydrating reagents conventionally used in organic chemistry and/or peptide synthesis. Non-limiting examples of the dehydrating reagent include diphenylphosphite, triphenylphosphite, N,N′-dicyclohexylcarbodiimide, N-(3-dimethylaminopropyl)-N′-ethylcarbodiimide hydrochloride, N,N′-diisopropylcarbodiimide and 1,1′-carbonyldiimidazole, N-methylimidazole, methane sulfony chloride, pivaloyl chloride and mixture thereof.

The term “cyclization reagent” comprises suitable cyclization reagent conventionally used in organic chemistry. Non-limiting examples of the cyclization reagent include Bis(trimethylsilyl)acetamide, Bis(trimethylsilyl)trifluoroacetamide, N-(Trimethylsilyl)acetamide and Hexamethyldisilazane.

In some embodiments, “X” comprises a halogen, mesylate, tosylate in the compound of formula-7 or formula-7′ and R₃ comprises an amino protective group in the compound of formula-7′, the compound of formula-8′ and the compound of formula 14. In preferred embodiment, “X” comprises Cl or Br in the compound of formula-7 or formula-7′; and R₃ comprises tetrahydropyranyl group in the compound of formula-7′, the compound of formula-8′ and the compound of formula 14.

In many cases, the compounds of the present invention are capable of forming acid addition salts by virtue of the presence of amino groups.

Acid addition salts may be prepared from inorganic acids or organic acids. The acid addition salts may be prepared from the inorganic acids selected from HCl, HBr, HF, H₂SO₄, HNO₃, H₃PO₄ and the like. The acid addition salts may be prepared from organic acids selected from acetic acid, propionic acid, glycolic acid, pyruvic acid, oxalic acid, malic acid, malonic acid, succinic acid, maleic acid, fumaric acid, tartaric acid, citric acid, benzoic acid, methanesulfonic acid, ethanesulfonic acid, p-toluene-sulfonic acid, salicylic acid and the like.

In some embodiments, the salt is a “pharmaceutically acceptable salt”. Exemplary pharmaceutically acceptable salts comprise acid addition salts of free bases formed with inorganic acids such as hydrochloric acid, hydrobromic acid, sulfuric acid, nitric acid, phosphoric acid.

The term “and/or” includes subject matter in the alternative as well as subject matter in combination. For instance, “x, and/or y”, includes “x or y” and “x and y”.

### Process:

In some embodiments, the present invention relates to a process for the preparation of a compound of formula-5

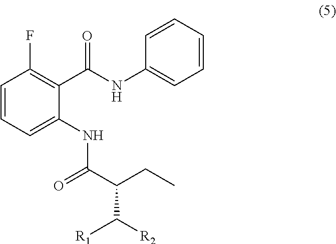

or a salt thereof, comprising
step a) reacting a compound of formula-3

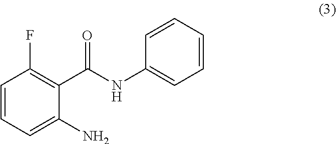 [SMILES: Nc1cccc(F)c1C(=O)Nc1ccccc1]

or a salt thereof, with a compound of formula-4

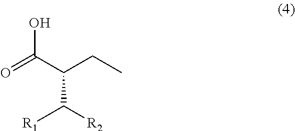

wherein, the compound of formula-5 or a salt thereof is obtained and wherein R₁ and R₂ comprise independently hydrogen and an amino protective group.

In some embodiments, the amino protective group comprises carbamate based amino protective groups, tetrahydropyranyl, alkylsilyl groups. In other embodiments, the amino protective group comprises carbamate. In some embodiments, the amino protective group comprises BOC.

In some embodiments, step a) comprises the step of including a dehydrating reagent in the reaction mixture. In some embodiments, step a) is performed in the presence of a dehydrating reagent. Non-limiting examples of the dehydrating reagent comprise DPP, TPP, DCC, EDC, and CDI, DIC, NMI, pivaloyl chloride, methane sulfonyl chloride or a mixture thereof.

In some embodiments, the dehydrating reagent comprises CDI. In some embodiments, step a) further comprises including CDI in the reaction mixture. In some embodiments, step a) is performed in the presence of CDI.

In some embodiments, step a) comprises the step of including a base in the reaction mixture. Non-limiting examples of the base includes pyridine, 4-dimethylaminopyridine, triethylamine, isopropylethylamine, imidazole, DABCO, DBU, 2,6-lutidine, N,N-diisopropylethylamine or a mixture thereof.

In some embodiments, step a) further comprises including a solvent in the reaction mixture; in another embodiment, the solvent comprises methanol, ethanol, isopropanol, n-propanol, acetonitrile, dimethylformamide, tert-butyl methyl ether, dichloromethane, ethyl acetate, isopropylacetate, toluene, 2-Methyltetrahydrofuran, diisopropylether, heptane, heptanes and combinations thereof.

In some embodiments, step a) comprises reacting a compound of formula-3 or a salt thereof with a compound of formula-4 or a salt thereof in the presence of a dehydrating reagent, a solvent, or a mixture thereof.

In some embodiments, step a) comprises reacting a compound of formula-3 or a salt thereof with a compound of formula-4 or a salt thereof in the presence of a dehydrating reagent, a base, a solvent or a mixture thereof.

In some embodiments, step a) is carried out at a temperature between about −80° C. and about 80° C., between about −40° C. and about 80° C., or between about −30° C. and and about 75° C. In some embodiments, step a) is performed at a temperature between about 50° C. and about 70° C. In some embodiments, step a) is performed at a temperature between about −40° C. and about −20° C.

In some embodiments, step a) comprising a step of isolation of the obtained solid of the compound formula-5, for example by rotary evaporation, spray drying, decantation, filtration, and centrifugation. In some embodiments, step a) comprising a step of isolation of the obtained solid of the compound formula-5 by filtration.

In some embodiments, step a) comprising a step of drying of the obtained solid under reduced pressure at between about 30° C. and about 80° C., or between about 40° C. and about 70° C. In some embodiments, step a) comprising a step of drying of the obtained solid under reduced pressure at between about 45° C. and about 65° C.

In some embodiments, the process further comprises step b) reacting the compound of formula-5 or a salt thereof, wherein at least one of R₁ and R₂ of the compound of formula-5 comprises an amino protective group with deprotection reagent to remove the amino protective group; wherein the compound of formula-6

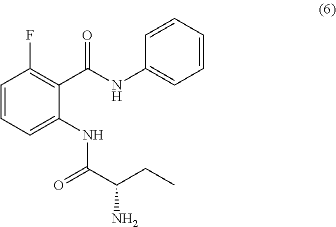 [SMILES: CC[C@H](N)C(=O)Nc1cccc(F)c1C(=O)Nc1ccccc1]

or a salt thereof is obtained.

In some embodiments, step b) comprises including deprotection reagent for the deprotection of amino protective groups from the amino group. For instance, if the amino protective group comprises a carbamate, such as BOC or FMOC, then the deprotection reagent is an acid. In further embodiments, the acid is a mineral acid. Non-limiting examples of mineral acids include HCl, HNO₃, H₃PO₄, H₂SO₄, H₃BO₃, HF, HBr, and HClO₄. In some embodiments, the deprotection reagent is HCl, HNO₃, H₃PO₄, H₂SO₄, H₃BO₃, HF, HBr and HClO₄, or a mixture thereof. In other embodiments, the acid is HCl. In a further embodiment, the acid comprises the acid generated in situ. For example, alcohol and acyl halide can be used to generate corresponding acid in situ. In one embodiment ethanol and acetyl chloride can be used to generate HCl in situ. In another example, if the amino protective group comprises an alkyl silyl group, the deprotection reagent is TBAF and/or TFA.

In some embodiments, step b) further comprises including in the reaction mixture a solvent; and the solvent comprises methanol, ethanol, isopropanol, n-propanol, acetonitrile, dimethylformamide, tert-butyl methyl ether, dichloromethane, ethyl acetate, isopropylacetate, toluene, 2-Methyltetrahydrofuran, diisopropylether, heptanes and combinations thereof.

In some embodiments, step b) is carried out at a temperature between about 0° C. and about 100° C.; between about 20° C. and about 90° C.; or between about 50° C. and about 80° C.

In some embodiments, the compound of formula-6 is prepared as the free base, whereas in other embodiments, the compound of formula-6 is prepared as a salt. In some specific embodiments, the compound of formula-6 is an HCl salt.

By way of example, preparation of the salt can be followed by a neutralization step to synthesize the free base.

In some embodiments, the process further comprises

step c) reacting the compound of formula-6 or a salt thereof with a compound of formula-7 or a compound of formula-7′

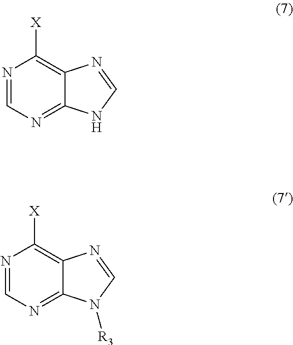

wherein, X comprises halogen, mesylate and tosylate and R₃ comprises an amino protective group and wherein a compound of formula-8 or formula-8′

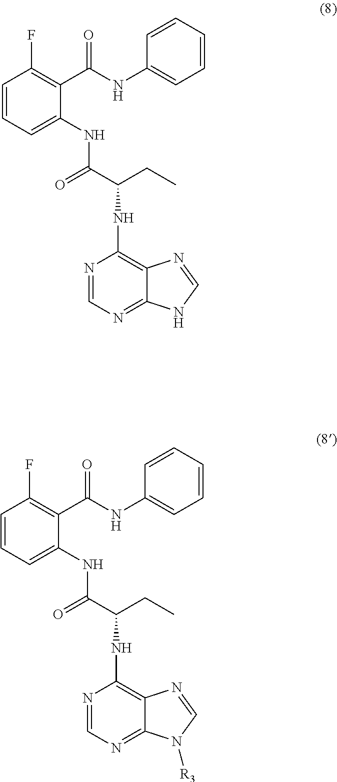

or a salt thereof is obtained.

In some embodiments, X comprises halogen. In other embodiments, X comprises Cl or Br. In other embodiments, X comprises Cl.

In some embodiments, step c) comprises a step of including a base in the reaction mixture. In another embodiment, the base comprises triethylamine, pyridine, isopropylethylamine, N,N-diisopropylethylamine, a carbonate base and combinations thereof.

In some embodiments, step c) comprises a step of including a catalyst in the reaction mixture. In other embodiment, the catalyst comprises tetrabutylammonium iodide, tetrabutylammonium bromide, tetrabutylammonium chloride and combinations thereof.

In some embodiments, step c) further comprises including a solvent in the reaction mixture. In other embodiment, the solvent comprises water, an alcoholic solvent, and combinations thereof and an alcoholic solvent comprises methanol, ethanol, isopropanol, n-propanol, t-butanol, isobutanol, n-butanol, pentanols, hexanols and combinations thereof.

In some embodiments, step c) comprises reacting the compound of formula-6 or a salt thereof with a compound of formula-7 or formula-7′ or a salt thereof in a solvent.

In some embodiments, step c) comprises reacting the compound of formula-6 or a salt thereof and a compound of formula-7 or formula-7′ or a salt thereof in presence of a base or a solvent.

In some embodiments, step c) is carried out at a temperature between about 10° C. and about 110° C.; between about 20° C. and about 100° C.; between about 50° C. and about 90° C.; or between about 60° C. and about 90° C.

In some embodiments, the compound of formula-8 or the compound of formula-8′ or a salt thereof is crystallized from solvent selected from the group comprising water, methanol, ethanol, isopropanol, n-propanol, acetonitrile, tert-butyl methyl ether, dichloromethane, ethyl acetate, isopropylacetate, toluene, 2-Methyltetrahydrofuran, diisopropylether and heptanes. In some embodiments, the compound of formula-8 or the compound of formula-8′ or a salt thereof is crystallized from solvent comprising acetonitrile, isopropanol, toluene, ethyl acetate and mixtures thereof.

In some embodiments, step c) comprising a step of isolation of the obtained solid of the compound formula-8 or formula 8′, for example by rotary evaporation, spray drying, decantation, filtration, and centrifugation. In some embodiments, step c) comprising a step of isolation of the obtained solid of the compound formula-8 or compound of formula 8′ by filtration.

In some embodiments, step c) comprising a step of drying of the obtained solid under reduced pressure at between about 30° C. and about 80° C. In some embodiments, step c) comprising a step of drying of the obtained solid under reduced pressure at between about 45° C. and about 65° C.

In one more embodiment, when compound of formula 8′ is obtained in the step c), the process further comprises step d) reacting the compound of formula-8′ or a salt thereof; with deprotection reagent to remove the amino protective group, wherein the compound of formula-8

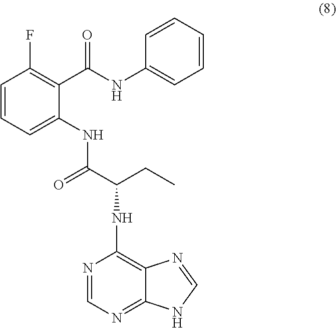 [SMILES: CC[C@H](Nc1ncnc2[nH]cnc12)C(=O)Nc1cccc(F)c1C(=O)Nc1ccccc1]

or a salt thereof is obtained.

In some embodiments, step d) comprises using deprotection reagent for the deprotection of amino protective groups. For instance, if the amino protective group comprises a carbamate, such as a BOC or FMOC, then the deprotection reagent comprises an acid. In further embodiments, the acid comprises a mineral acid. Non-limiting examples of mineral acids include HCl, HNO₃, H₃PO₄, H₂SO₄, H₃BO₃, HF, HBr and HClO₄. In some embodiments, the deprotection reagent comprises HCl, HNO₃, H₃PO₄, H₂SO₄, H₃BO₃, HF, HBr, HClO₄ or a mixture thereof. In other embodiments, the acid comprises HCl. In another example, if the amino protective group comprises an alkyl silyl group, the deprotection reagent comprises TBAF and/or TFA.

In some embodiments, the amino protective group R₃ comprises THP and the deprotection reagent to remove the amino protective group comprise an acid. In a further embodiment, the acid is generated in situ. For example, alcohol and acyl halide can be used to generate corresponding acid in situ. In one embodiment ethanol and acetyl chloride can be used to generate HCl in situ. In another embodiment methanol and acetyl chloride can be used to generate HCl in situ. In some embodiments, step d) comprises an acid selected from the group consisting of a mineral acid, TFA and a Lewis acid. In some embodiments the acid comprises HCl.

In some embodiments, step d) is carried out in solvent selected from the group comprising water, methanol, ethanol, isopropanol, n-propanol, acetonitrile, tert-butyl methyl ether, dichloromethane, ethyl acetate, isopropylacetate, toluene, 2-Methyltetrahydrofuran, diisopropylether, heptanes and mixture thereof.

In some embodiments, step d) is carried out at a temperature between about 30° C. and about 70° C.; between about 40° C. and about 60° C.; or between about 25° C. and about 50° C.

In some embodiments, the compound of formula-8 is obtained as the free base, whereas in other embodiments, the compound of formula-8 is obtained as a salt. In some embodiments, the compound of formula-8 is HCl salt.

By way of example, preparation of the salt can be followed by a neutralization step to synthesize the free base.

In some embodiments, the process further comprises step e) comprising a step of reacting the compound of formula-8 or a salt thereof with cyclization reagent to cyclize the compound of formula-8, wherein the compound of formula-1

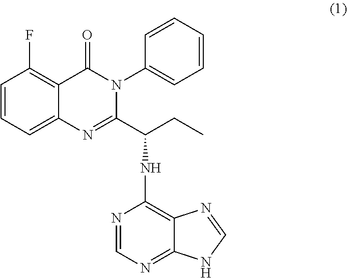 [SMILES: CC[C@H](Nc1ncnc2[nH]cnc12)c1nc2cccc(F)c2c(=O)n1-c1ccccc1]

or a salt thereof is obtained.

In some embodiments, step e) comprises a step of using a cyclization reagent. In some embodiments, step e) is performed in the presence of a cyclization reagent. Non-limiting examples of the cyclization reagent include Bis(trimethylsilyl)acetamide, Bis(trimethylsilyl)trifluoroacetamide, N-(Trimethylsilyl)acetamide and Hexamethyldisilazane. In some embodiments, step e) comprises using Bis(trimethylsilyl)acetamide. In some embodiments, step e) is performed in the presence of Bis(trimethylsilyl)acetamide.

In some embodiments, step e) optionally comprises use of catalyst. In other embodiment the catalyst is selected from iodine or a fluoride ion catalyst such as tetarbutyl ammonium fluoride.

In some embodiments, step e) comprises reacting a compound of formula-8 or a salt thereof with a cyclization reagent, in the presence of at least one solvent.

In some embodiments, step e) is carried out at a temperature between about 20° C. and about 200° C.; between about 60° C. and about 180° C.; or between about 30° C. and about 100° C. or between about 100° C. and about 160° C.

In some embodiments, the compound of formula-1 or a salt thereof is crystallized from solvent selected from the group comprising of water, methanol, ethanol, isopropanol, n-propanol, acetonitrile, tert-butyl methyl ether, dichloromethane, ethyl acetate, isopropylacetate, toluene, 2-methyltetrahydrofuran, diisopropylether and heptanes. In some embodiments, the compound of formula-1 or a salt thereof is crystallized from the solvent comprising water, methanol, ethanol, propanol, butanol, acetonitrile and mixtures thereof.

In some embodiments, step e) comprising a step of isolation of the obtained solid of the compound formula-1, for example by rotary evaporation, spray drying, decantation, filtration, and centrifugation. In some embodiments, step e) comprising a step of isolation of the obtained solid of the compound formula-1 by filtration.

In some embodiments, step e) comprising a step of drying of the obtained solid under reduced pressure at between about 30° C. and about 80° C. In some embodiments, step e) comprising a step of drying of the obtained solid under reduced pressure at between about 45° C. and about 65° C.

In one more embodiment, the present invention alternatively relates to a process for the preparation of a compound of formula-1

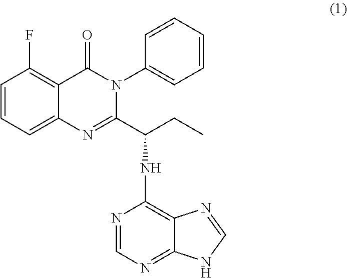 [SMILES: CC[C@H](Nc1ncnc2[nH]cnc12)c1nc2cccc(F)c2c(=O)n1-c1ccccc1]

or a salt thereof, comprising
step (i) reacting the compound of compound of formula-8′

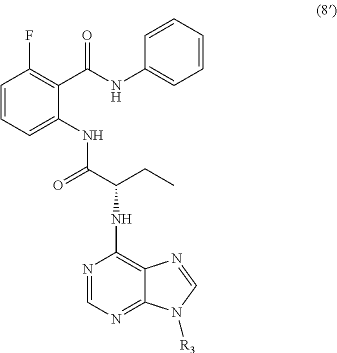

or a salt thereof with cyclization reagent to cyclize the compound of formula-8′, wherein a compound of formula-14

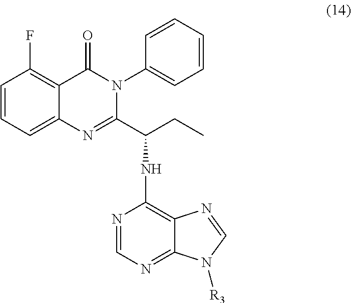

or a salt thereof is obtained; and
step (ii) reacting the compound of formula-14 or a salt thereof with deprotection reagent to remove the amino protective group, wherein the compound of formula-1 is obtained.

In some embodiments, step (i) comprises using a cyclization reagent. In some embodiments, step (i) is performed in the presence of a cyclization reagent. Non-limiting examples of the cyclization reagent include Bis(trimethylsilyl)acetamide, Bis(trimethylsilyl)trifluoroacetamide, N-(Trimethylsilyl)acetamide and Hexamethyldisilazane. In some embodiments, step (i) comprises using Bis(trimethylsilyl)acetamide. In some embodiments, step (i) is performed in the presence of Bis(trimethylsilyl)acetamide.

In some embodiments, step (i) optionally comprises a step of using catalyst. In other embodiment the catalyst is selected from iodine or a fluorine ion catalyst such as tetarbutyl ammonium fluoride.

In some embodiments, step (ii) comprises using deprotection reagent for the deprotection of amino protective groups. For instance, if the amino protective group comprises a carbamate, such as a BOC or FMOC, then the deprotection reagent comprises an acid. In further embodiments, the acid comprises a mineral acid. Non-limiting examples of the mineral acids include HCl, HNO₃, H₃PO₄, H₂SO₄, H₃BO₃, HF, HBr and HClO₄. In some embodiments, the reagent comprises HCl, HNO₃, H₃PO₄, H₂SO₄, H₃BO₃, HF, HBr, HClO₄ or a mixture thereof. In other embodiments, the acid comprises HCl. In another example, if the amino protective group comprises an alkyl silyl group, the deprotection reagent comprises TBAF and/or TFA.

In some embodiments, the amino protective group R₃ comprises THP and the deprotection reagent to remove the amino protective group comprise an acid. In a further embodiment, the acid comprises the acid, which is generated in situ. For example, methanol and acyl halide can be used to generate corresponding acid in situ. In one embodiment ethanol and acetyl chloride can be used to generate HCl in situ. In another embodiment methanol and acetyl chloride can be used to generate HCl in situ. In some embodiments, step ii) comprises an acid selected from the group consisting of a mineral acid, TFA and a Lewis acid. In some embodiments the acid comprises HCl.

In some embodiments the compound of formula-14 is optionally isolated. In some embodiments, the compound of formula-14 is converted into compound of formula-1 in situ.

In some embodiments, step (ii) is carried out at a temperature between about 30° C. and about 70° C.; between about 40° C. and about 60° C.; or between about 25° C. and about 50° C.

By way of example, the resulting compounds from the processes described herein may be used in a pharmaceutical composition. In another embodiment, provided is a pharmaceutical composition comprising a resulting compound from the processes disclosed herein or a salt thereof, and one or more pharmaceutically acceptable carriers or excipients.

Present invention is further illustrated with the following non-limiting examples.

### Examples

### Example-1: Preparation of 2-amino-6-fluoro-N-phenylbenzamide

To solution of 2-fluoroanthranilic acid (100 gm, 0.65 moles) in DMF (220 ml), CDI (125.43 gm, 0.78 moles) was added. The reaction mass was heated at 40-50° C. and stirred for 3-6 hours. The reaction mass was cooled to 25-35° C. and aniline (79 gm, 0.85 moles) was added to the reaction mass and stirred for 2-4 hours at 25-35° C. The reaction mass was quenched in to water (2500 ml) and stirred for 12-16 hours. Precipitated solid was separated by filtration, washed with water (5×1000 ml) and dried under reduced pressure to get off white solid (124.0 gm).

H¹ NMR (CDCl₃) δ (ppm): 5.97 (2H, NH₂), 6.405-7.624 (8H, Aromatic-H), 8.323-8.362 (1H, NH).

Mass (M+1): 230.95.

### Example-2: Preparation of (S)-tert-butyl(1-((3-fluoro-2-(phenylcarbamoyl) phenyl) amino)-1-oxobutan-2-yl)carbamate

To solution of (S)-2-((tert-butoxycarbonyl)amino)butanoic acid (68 gm) in DMF (120 ml), CDI (67.6 gm) was added at 25-35° C. and stirred for 2 hours. A solution of 2-amino-6-fluoro-N-phenylbenzamide (50 gm) in DMF (60 ml) was slowly added over 30 min. to the reaction mixture. The reaction mixture was then heated at 50-60° C. for 36-48 hours. After completion of reaction, the reaction mixture was slowly added to water (1500 ml). The precipitated solid was stirred for 4-5 hours. The solid was then filtered, washed with water and dried under reduced pressure at 50-55° C. to get the title compound (72 gm).

¹H NMR (CDCl₃) δ (ppm): 1.035 (3H), 1.144-1.534 (9H), 1.711-1.800 (2H), 5.139-5.157 (1H), 6.898-8.402 (8H), 8.402-8.437 (1H), 8.523 (1H), 11.722 (1H).

Mass (M+1): 416.25.

### Example-3: Preparation of (S)-tert-butyl(1-((3-fluoro-2-(phenylcarbamoyl) phenyl) amino)-1-oxobutan-2-yl)carbamate

(S)-2-((tert-butoxycarbonyl)amino)butanoic acid (124 gm) was added to dichloromethane (500 ml) at 20-30° C. and the obtained solution was cooled to −35° C. N-methylimidazole (143 gm) was added to the solution followed by the addition of methane sulfonyl chloride solution in dichloromethane−30 to −35° C. A solution of 2-amino-6-fluoro-N-phenylbenzamide (100 gm) in dichloromethane (60 ml) was slowly added over 30 min at −30 to −35° C. to the reaction mixture. After completion of the reaction, water (800 ml) was added to the reaction mixture. The organic layer was separated and the aqueous layer was washed with dichloromethane. Both the dichloromethane layers were combined and concentrated under vacuum at 45° C. up to 2.5-3.5 volume. Isopropanol (600 ml) was added to the reaction mass and the isopropanol was recovered under vacuum. The reaction mass was filtered and washed twice with isopropanol (50 ml). Obtained solid was dried under vacuum at 50-55° C. to get the title compound (135 gm).

### Example-4: Preparation of (S)-2-(2-aminobutanamido)-6-fluoro-N-phenyl-benzamide

To a solution of (S)-tert-butyl (1-((3-fluoro-2-(phenylcarbamoyl)phenyl)amino)-1-oxobutan-2-yl)carbamate (85 gm) in methanol (425 ml), concentrated HCl (85 ml) was added drop wise within 30 min. The reaction mixture was then heated to 65-75° C. for 12 hours and concentrated. Ethyl acetate (255 ml) was added to the reaction mass and stirred for 1-2 hours. The reaction mixture was cooled at 5-10° C. and stirred for 1-2 hours. Precipitated solid was filtered and washed with cooled ethyl acetate (40 ml). To a round bottom, wet solid, water (255 ml) and ethyl acetate (255 ml) were added. 15% sodium carbonate solution was used to adjust pH to 8-9 of the reaction mixture. Organic layer was separated and concentrated under reduced pressure to get the title compound (33 gm).

¹H NMR (MeOD) δ (ppm): 0.960-0.922 (t, 3H), 1.549-1.855 (m, 2H), 3.382-3.304 (m, 1H), 7.001-7.981 (m, 8H).

Mass (M+1): 316.

### Example-5: Preparation of (S)-2-(2-((9H-purin-6-yl)amino)butanamido)-6-fluoro-N-phenylbenzamide

To a solution of (S)-2-(2-aminobutanamido)-6-fluoro-N-phenylbenzamide (7 gm, 0.0222 moles) and 6 chloro purine (4.4 gm, 0.0288 moles) in tert-butanol (70 ml), tetrabutylammonium iodide (3.5 gm 0.0094 moles) and diisopropylethylamine (7.7 ml, 0.0444 moles) were added. The reaction mixture was heated at 80 to 85° C. for 45-50 hours. The reaction mixture was then concentrated. Ethyl acetate (70 ml) and water (70 ml) were added and stirred 20-30 min Organic layer was separated organic layer. The aqueous layer was extracted by ethyl acetate (70 ml). Both organic layers were mixed and washed by water (50 ml). Concentrate organic layer under reduced pressure and stripped by toluene (50 ml). Ethyl acetate (21 ml) was added to the reaction mass and stirred. The reaction mixture was cooled at 10° C., filtered and washed by chilled ethyl acetate (7 ml). The filtered product was dried under reduced pressure at 45 to 50° C. to get yellow solid (4 gm).

¹H NMR (MeOD) δ (ppm): 1.145-1.108 (t, 3H), 2.186-1.950 (m, 2H), 4.631 (s, 1H), 8.145-6.971 (m, 10H).

Mass (M+1): 434.

### Example-6: Preparation of 2-fluoro-N-phenyl-6-((2S)-2-((9-(tetrahydro-2H-pyran-2-yl)-9H-purin-6-yl) amino)butanamido)benzamide

To a solution of (S)-2-(2-aminobutanamido)-6-fluoro-N-phenylbenzamide (13 gm) in ethanol (30 ml), 6-chloro-9-(tetrahydro-2H-pyran-2-yl)-9H-purine (11.7 gm), triethyl amine (8.6 ml) and water (30 ml) were added and heated to 75-85° C. The reaction mixture was stirred for 24-30 hours at 75-85° C. The reaction mixture was then cooled to 25-35° C. and extracted with ethyl acetate (100 ml×2). Both ethyl acetate layers were mixed and concentrated. Toluene (30 ml) was added to the residue. The reaction mixture was cooled at 5-10° C. and stir for 2 hours at 5-10° C. Precipitated solid was filtered and washed with toluene (10 ml). The solid was dried under reduced pressure at 40-50° C. to get the title compound (11 gm).

¹H NMR (CDCl₃) δ (ppm): 1.093 (m, 3H), 1.980-2.309 (m, 8H), 3.710-4.160 (m, 2H), 4.631 (s, 1H), 5.494-5.563 (t, 1H), 6.951-8.723 (m, 11H), 11.00 (s, 1H).

Mass (M+1): 518.

### Example-7: Preparation of (S)-2-(2-((9H-purin-6-yl)amino)butanamido)-6-fluoro-phenylbenzamide

To a solution of 2-fluoro-N-phenyl-6-((2S)-2-((9-(tetrahydro-2H-pyran-2-yl)-9H-purin-6-yl)amino)butanamido) benzamide (2 gm) in ethanol (20 ml), acetyl chloride (0.5 ml) was slowly added at 25-35° C. The reaction mixture was stirred for 2-3 hours at 25-35° C. After completion of reaction, water (2 ml) and a solution of 5% sodium bicarbonate (50 ml) was added drop wise. The reaction mixture was stirred and the precipitated product was filtered. The precipitated product was washed with water (20 ml) and dried under reduced pressure at 45-50° C. to get the title compound (1.45 gm).

¹H NMR (MeOD) δ (ppm): 1.145-1.108 (t, 3H), 2.186-1.950 (m, 2H), 4.631 (s, 1H), 8.145-6.971 (m, 10H).

Mass (M+1): 434.

### Example-8: Preparation of Idelalisib

To a solution of (S)-2-(2-((9H-purin-6-yl)amino)butanamido)-6-fluoro-N-phenylbenzamide (1 gm) in Bis(trimethylsilyl)acetamide (15 ml), Catalytic iodine was added. The reaction mixture was heated at 140-150° C. and stirred for 7-9 hours at 140-150° C. The reaction mass was concentrated under reduced pressure and stripped by toluene (20 ml). Ethanol (20 ml) and charcoal (0.1 gm) were added to the reaction mass and stirred 20-30 min. The reaction mass was filtered and concentrated. Methanol (5 ml) was added to the reaction mass and stirred 20-30 min. the reaction mass was then added to water (40 ml) and stirred for 1-2 hours. The precipitated product was filtered and dried under reduced pressure at 50-55° C. to get the title compound (0.8 gm).

¹H NMR (DMSO-d6) δ (ppm): 0.751-0.716 (t, 3H), 1.907-1.753 (s, 2H), 4.650 (s, 1H), 8.268-7.248 (m, 11H).

Mass (M+1): 416.

### Example-9: Preparation of Idelalisib

2-Fluoro-N-phenyl-6-((2S)-2-((9-(tetrahydro-2H-pyran-2-yl)-9H-purin-6-yl)amino) butanamido) benzamide (2 gm) was added to Bis(trimethylsilyl)acetamide (20 ml) at 25° C. and then catalytic amount of iodine was added to the obtained reaction mass. The reaction mass was heated to 140° C. and stirred for 5-7 hours. The obtained reaction mass was concentrated at 40-50° C. and stripped with toluene at 40-50° C. Isopropanol (40 ml) was charged into the obtained mass and treated with activated charcoal. The obtained mass was charged into MTBE (20 ml) and ethanol (40 ml) and stirred for 6-9 hours and then cooled to 5-10° C. The precipitated mass was filtered and washed with chilled MTBE (10 ml) at 5-10° C. to get wet solid. The obtained wet solid was charged into ethanol and heated to 70° C. and stirred for 2-4 hours at 25-30° C. to obtain a slurry. The obtained wet mass was filtered and dried at 50° C. under vacuum to get title compound (0.75 gm).

1: A process for the preparation of the compound of formula-1 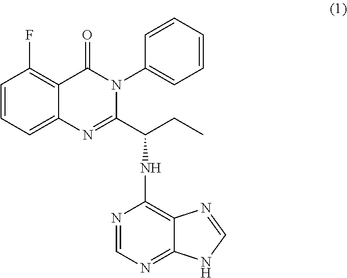 [SMILES: CC[C@H](Nc1ncnc2[nH]cnc12)c1nc2cccc(F)c2c(=O)n1-c1ccccc1]
or a salt thereof; comprising reacting a compound of formula-6 or a salt thereof 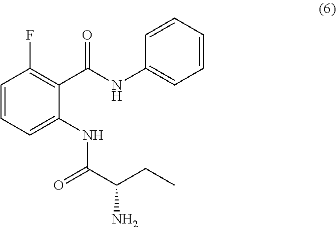 [SMILES: CC[C@H](N)C(=O)Nc1cccc(F)c1C(=O)Nc1ccccc1]
with a compound of formula-7 or a compound of formula-7′ 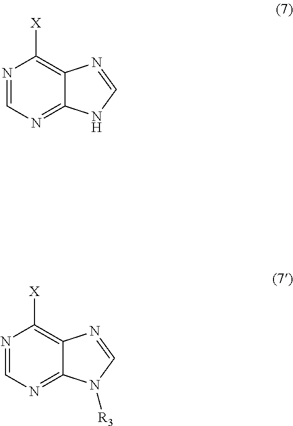
wherein X is halogen, mesylate or tosylate and R₃ is an amino protective group.

2: A process for the preparation of compound of formula-1 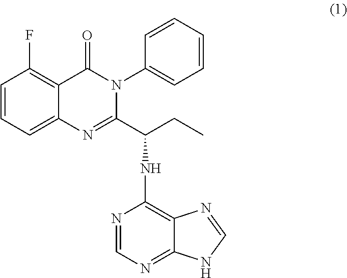 [SMILES: CC[C@H](Nc1ncnc2[nH]cnc12)c1nc2cccc(F)c2c(=O)n1-c1ccccc1]
or a salt thereof; comprising steps of:
a) reacting a compound of formula-6 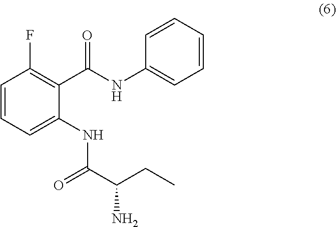 [SMILES: CC[C@H](N)C(=O)Nc1cccc(F)c1C(=O)Nc1ccccc1]
or a salt thereof, with a compound of formula-7 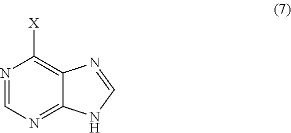
wherein X is halogen, mesylate and tosylate
to obtain a compound of formula-8 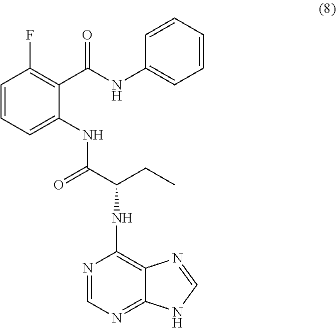 [SMILES: CC[C@H](Nc1ncnc2[nH]cnc12)C(=O)Nc1cccc(F)c1C(=O)Nc1ccccc1]
or salts thereof; and
b) reacting compound of formula-8 or a salt thereof with cyclization reagent to cyclize the compound of formula-8, to obtain compound of formula-1.

3: A process for the preparation of compound of formula-1 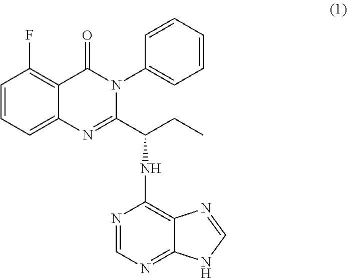 [SMILES: CC[C@H](Nc1ncnc2[nH]cnc12)c1nc2cccc(F)c2c(=O)n1-c1ccccc1]
or a salt thereof; comprising steps of:
a) reacting a compound of formula-6 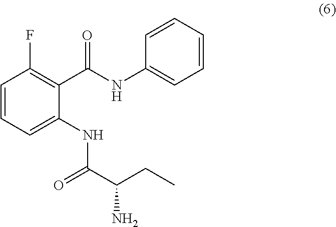 [SMILES: CC[C@H](N)C(=O)Nc1cccc(F)c1C(=O)Nc1ccccc1]
or a salt thereof, with a compound of formula-7′ 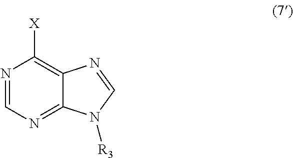
wherein X is halogen, mesylate and tosylate and R₃ is an amino protective group to obtain a compound of formula-8′ 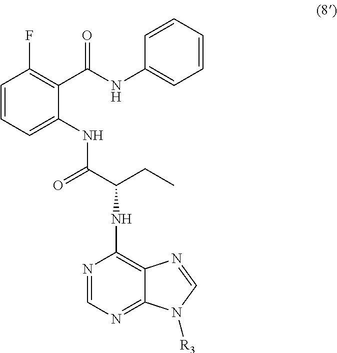
or salts thereof; and
b) reacting the compound of formula-8′ or a salt thereof with deprotection reagent to remove the amino protective group to obtain compound of formula-8, 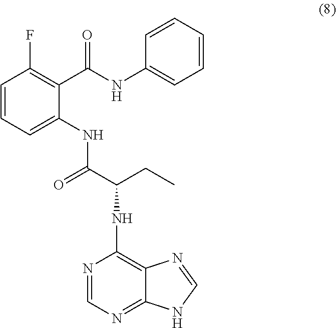 [SMILES: CC[C@H](Nc1ncnc2[nH]cnc12)C(=O)Nc1cccc(F)c1C(=O)Nc1ccccc1]
or salt thereof; and
c) reacting compound of formula-8 or a salt thereof with cyclization reagent to cyclize the compound of formula-8, to obtain compound of formula-1.

4: The process according to claim 1, further comprising preparation of compound of formula-6 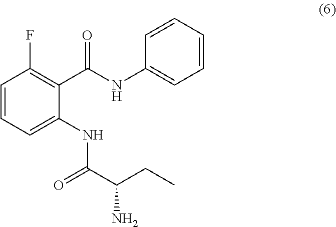 [SMILES: CC[C@H](N)C(=O)Nc1cccc(F)c1C(=O)Nc1ccccc1]
comprising:
a) reacting a compound of formula-3 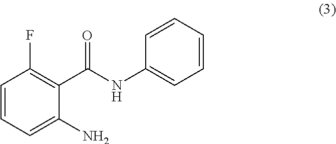 [SMILES: Nc1cccc(F)c1C(=O)Nc1ccccc1]
or a salt thereof with a compound of formula-4 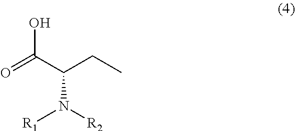
wherein R₁ and R₂ are independently hydrogen and an amino protective group to obtain compound of formula-5, 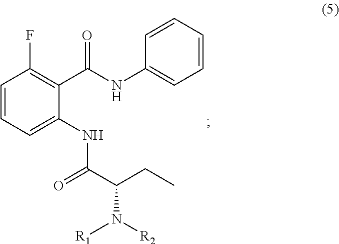
or a salt thereof; and
b) when at least one of R₁ and R₂ is amino protective group, reacting the compound of formula-5 with deprotection reagent to remove the amino protective group to obtain compound of formula-6.

5: The process according to claim 1, wherein the amino protective group is selected from methyl carbamate, 9-fluorenylmethyl carbamate, 2,2,2-trichloroethyl carbamate, 2-trimethylsilylethyl carbamate, 1,1-dimethylpropynyl carbamate, t-butyl carbamate, vinyl carbamate, allyl carbamate, t-butyl carbamate, tetrahydropyranyl and alkylsilyl group.

6: The process according to claim 5, wherein the amino protective group is tetrahydropyranyl group.

7: The process according to claim 1, wherein the reaction of compound of formula-6 with a compound of formula-7 or a compound of formula-7′ is carried out in presence of a base, wherein the base is selected from pyridine, 4-dimethylaminopyridine, triethylamine, isopropylethylamine, imidazole, 1,4-diazabicyclo[2.2.2]octane, 1,8-Diazabicyclo[5.4.0]undec-7-ene, 2,6-lutidine, N,N-diisopropylethylamine and a mixture thereof.

8: The process according to claim 1, wherein the reaction of compound of formula-6 with a compound of formula-7 or a compound of formula-7′ is carried out in presence of dehydrating reagent, wherein the dehydrating reagents is selected from diphenylphosphite, triphenylphosphite, N,N′-dicyclohexylcarbodiimide, N-(3-dimethylaminopropyl)-N′-ethyl-carbodiimide hydrochloride, N, N′-diisopropylcarbodiimide, 1,1′-carbonyldiimidazole, N-methyl imidazole, pivaloyl chloride, methane sulfonyl chloride and mixtures thereof.

9-12. (canceled)

13: The process according to claim 3, wherein the deprotection reagent is selected from hydrochloric acid, nitric acid, phosphoric acid, sulfuric acid, boric acid, hydrofluoric acid, hydrobromic acid and perchloric acid.

14: The process according to claim 2, wherein the cyclization reagent is selected from Bis(trimethylsilyl)acetamide, Bis(trimethylsilyl) trifluoroacetamide, N-(Trimethylsilyl)acetamide and Hexamethyldisilazane.

15-17. (canceled)

18: The process according to claim 3, wherein the cyclization reagent is selected from Bis(trimethylsilyl)acetamide, Bis(trimethylsilyl) trifluoroacetamide, N-(Trimethylsilyl)acetamide and Hexamethyldisilazane.

19: The process according to claim 1, wherein the deprotection reagent is selected from hydrochloric acid, nitric acid, phosphoric acid, sulfuric acid, boric acid, hydrofluoric acid, hydrobromic acid and perchloric acid

20: A compound selected from

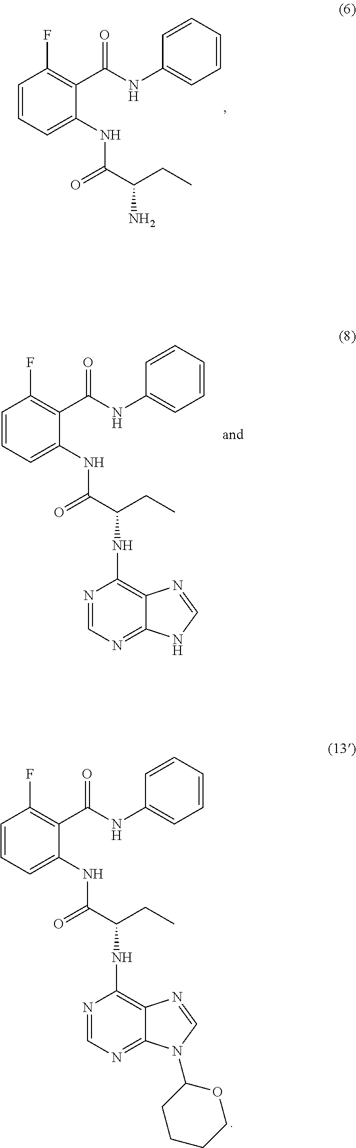 [SMILES: CC[C@H](N)C(=O)Nc1cccc(F)c1C(=O)Nc1ccccc1 ; CC[C@H](Nc1ncnc2[nH]cnc12)C(=O)Nc1cccc(F)c1C(=O)Nc1ccccc1 ; CC[C@H](Nc1ncnc2c1ncn2C1CCCCO1)C(=O)Nc1cccc(F)c1C(=O)Nc1ccccc1]

21-24. (canceled)
